(19) United States
(12) Patent Application Publication
(10) Pub. No.: US 2015/0306546 A1
(43) Pub. Date: Oct. 29, 2015

(54) METHOD FOR OBTAINING POROUS HOLLOW FIBERS HAVING MOLECULARLY IMPRINTED SPHERES EMBEDDED THEREIN AND MODULAR DEVICE FORMED BY THE RESULTING FIBERS

(71) Applicant: INSTITUTO TECNOLÓGICO Y DE ESTUDIOS SUPERIORES DE MONTERREY, Monterey, Nuevo León (MX)

(72) Inventors: Dennise Alejandra MURGUIA FLORES, Monterrey (MX); Jaime BONILLA RIOS, Monterrey (MX); Jorge Armando CORTES RAMIREZ, Monterrey (MX)

(21) Appl. No.: 14/648,512

(22) PCT Filed: Nov. 28, 2013
(86) PCT No.: PCT/MX2013/000152

§ 371 Date: May 29, 2015

(30) Foreign Application Priority Data
Nov. 30, 2012 (MX) MX/A/2012/013984

(51) Int. Cl. B01D 69/08; B29C 53/32; A61M 1/16; B01D 63/02; B01D 71/68; B29C 47/00; B01D 61/24

(52) U.S. Cl. CPC B01D 69/087; B29C 47/0014; B29C 47/0026; B29C 53/32; B01D 61/246; B01D 63/02; B01D 71/68; A61M 1/16; B29L 2023/00

(57) ABSTRACT

The invention relates to a method for obtaining porous hollow fibers having molecularly imprinted spheres embedded therein, said method comprising the steps of: simultaneously and independently injecting a doping solution and a pore-forming solution into an extrusion die; extruding or spinning porous hollow fibers; coagulating the porous hollow fibers that leave step (b) in an aqueous solution with an aqueous solution having a temperature between 15° C.-25° C.; winding the flexible porous hollow fibers coagulated in step (c); leaving them to dry and storing same under vacuum conditions. The resulting porous hollow fibers are used to form filtrating modules in which a plurality of fibers are disposed longitudinally and in parallel. The filtrating modules can be assembled together to form filtering devices intended, in a preferred example, for gradual blood purification and the subsequent qualitative and/or quantitative identification of the molecules retained in the first filter of each filtering module. The invention also relates to the operating method of the filtering device.

This patent application is a national phase filing under section 371 of PCT/MX2013/000152, filed Nov. 28, 2013, which claims the priority of Mexican patent application MX/a/2012/013984, filed Nov. 30, 2012, each of which is incorporated herein by reference in its entirety.

### TECHNICAL FIELD

The present application for patent of invention aims to provide a process for obtaining porous hollow fibers or fibers with molecularly imprinted spheres therein and a modular device made by the resulting fibers.

### BACKGROUND

To carry out the hemopurification processes, devices formed by natural polymer matrices are known such as starch, cellulose, cellulose triacetate, that the material which they are formed offer advantages of biocompatible, non-toxic neutral matrices. However, they have the limitation that devices formed by them lack a mechanical stability with a continuous flow passing through it.

Devices are also known formed by synthetic matrices as polyether oxide, polysulfones, SPAN-80, polymethyl methacrylate, polyacrylonitrile; provide devices with a higher mechanical stability than devices formed by natural matrices. However, their hydrophilicity to the molecules of the blood proteins is limited.

As examples of synthetic devices it can mention:

That disclosed in JP2006230459A2 entitled POLYSULFONE HOLLOW FIBER MEMBRANE BUNDLE HAVING PERMSELECTIVITY AND BLOOD PURIFIER. This module of porous polysulfone Membranes provides Permeo-selectivity and containing polyvinylpyrrolidone (PVP). The present invention proposes to provide a process for obtaining hollow fibers with molecularly imprinted spheres embedded therein, comprising a natural matrix such as molecularly imprinted spheres with a synthetic matrix in the form of hollow porous fibers. The present invention has a modular device formed by the resulting fibers, wherein said fibers allow to have a higher mechanical stability than those of commercially available synthetic fibers, because in its interior comprises molecularly imprinted spheres, whereby it is possible to have a higher stability thereof to the passage of fluid; added to this, the conformation described allows to increase the internal surface area, thus achieving a higher retention of molecules of interest. As compared to patent JP2006230459A2, it is only given by a single module.

In the present invention, the modular device formed by hollow porous fibers embedded with molecularly imprinted spheres has structural stability and gives a superior support to the fibers, from the membrane embedded with spheres.

### BRIEF DESCRIPTION OF THE DRAWINGS

FIG. 1. Block diagram of the process of obtaining porous hollow fibers with molecularly imprinted spheres embedded therein.

FIG. 2. Schematic representation of the process of obtaining hollow porous fibers with molecularly imprinted spheres embedded therein.

FIG. 3. Schematic representation of a Module formed by the resulting fibers by the obtainment process proposed.

FIG. 4. Schematic representation of the modular device reason of this patent application.

### DETAILED DESCRIPTION

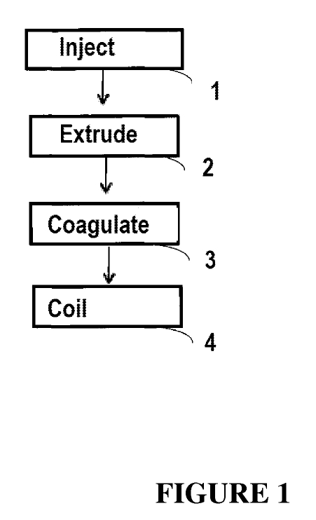

The present patent application provides a process for obtaining porous hollow fibers with and molecularly imprinted spheres embedded therein, for filtering and/or separation of macromolecules suspended in liquids, the process is represented in block diagram in FIG. 1, and comprising the steps of:
a) Injecting (1) simultaneously and independently a doping solution and a pore-forming solution in an extrusion die.

The doping solution contains a plurality of molecularly imprinted microspheres dissolved in a solvent such as: n-methyl pyrrolidone, concentrated or dilute acetone between 1-2 M and possible combinations between them.

The pore-forming solution comprising Polyethersulfone (PES) and polyether glycol (PEG) dissolved in a ratio ranging from 1:2 to 1:3.

Each of the solutions is stored independently and its inflow in the extruder die (5) is regulated by pumps (6) so that it reaches the ideal operating conditions of temperature in the range of 40° C.-70° C. and 20-30 Pascal of pressure for extrusion conditions on the die. The inflow is preferred in the range of 3-5 mL/sec,
b) Extruding (2) or spinning hollow porous fibers with an outer diameter of 0.72 and an inner diameter of 0.26 mm with an outflow equal to the injection flow of step a).
c) Coagulating (3) the hollow porous fibers coming from step b in aqueous solution, this coagulation stage is performed in a container (7) with an aqueous solution, preferably water, which is at a temperature in the range of 15° C.-25° C.
d) Coiling (4) flexible and hollow porous fibers coagulated in the step c), on a reel (8); let them dry and store in vacuum conditions.

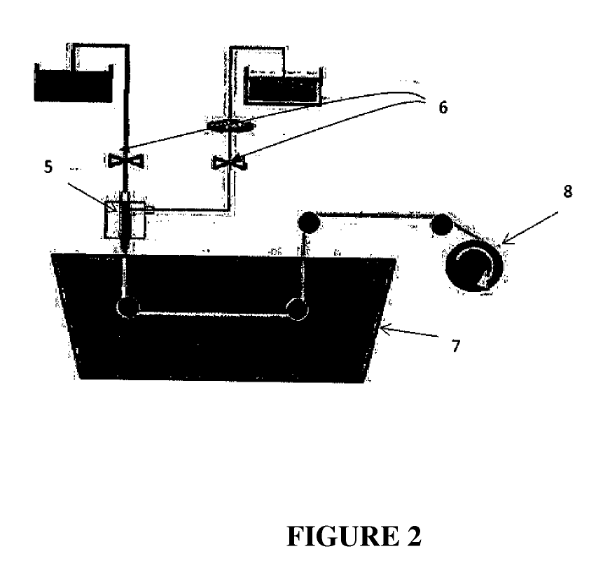

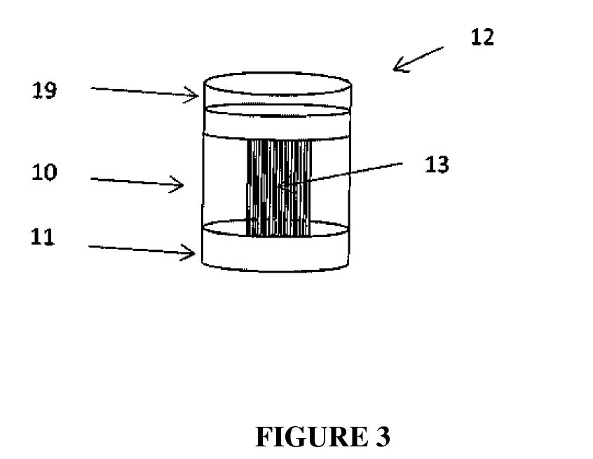

In FIG. 2, is shown a schematic representation of the devices to which the above process preferably is implemented.

It is important to note that the resulting hollow porous fibers increase the purification of a particular sample, because they have the capacity to retain a molecule of interest, because this increases the internal surface, and this encourages a higher fluid contact and thus an increased retention of said molecule.

Further, the microspheres embedded in the fibers will provide rigidity to the fiber, which resists passage of a fluid without suffering folds.

It is important to note that elongation and tensile strength tests of the fibers were determined with and without embedded microspheres. See Table 1.

The tests of elongation and tension of the fibers with molecularly imprinted spheres embedded were carried out using samples (50×10 mm) in LTS-500N-S20 tensile and compression test machine (Minebea, Kanagawa, Japan) which is a universal measuring machine equipped with a cell of 2.5 kN. The strips 50 mm length and 10 mm wide were cut from the principal samples with a cutter. A sample set, for those having molecularly imprinted spheres and do not, and each set was repeated 3 times. Only samples in which the cutting was made near the length measurement were considered for calculation of tensile strength. The value of the tensile strength (N/mm2) and the elongation (%) were calculated using the following equations:

$$tensile strength = \frac{maximum load}{diagonal area}$$

$$elongation = [ \frac{manixum length in maximum load}{initial length} ] \times 100$$

With the results obtained, it is shown that the fibers with embedded micro spheres give a higher stability to the fibers, which increases their deformation capability and prevents the fracture thereof during the passage of the sample.

TABLE 1
Results of Tensile Strength and Elongation tests.

| | | Fibers without |
|---|---|---|
| | Fibers embedded in | molecularly imprinted |
| | microspheres | microspheres |
| Elongation (%) | 37.6 ± 0.5 | 32.3 ± 3.7 |
| Tensile strength (N/mm²) | 64.5 ± 1.5 | 62 ± 3 |

With the resulting hollow fibers with the above described process, a filtering Module (12) formed by:

A first filter (9) closing the upper end of a cylindrical container (10) that in its interior comprises a plurality of parallel hollow porous fibers and arranged longitudinally, and closing the lower end of the container is a second filter (11).

Particularly, the first filter (9) has a higher pore size than the pore size of the second filter (11), so that by passing a solution containing macromolecules suspended by the module (12), the larger macromolecules than the pore size of the filter are retained, and the smaller ones pass, being retained those having a size that matches the pores of the fibers and the size of the imprints of the spheres, and the smaller ones are released and leave the second filter (11) whose pore size is less than the pore size of the hollow fiber and molecularly imprinted spheres embedded therein. In another preference, the pore size of the first (9) and second filter (11) of the same module (12) may be the same, and since that the modules (12) are coupled together, the second filter (11) of a Module corresponds to the first filter (9) of a Module that follows it.

Note that the size of the pores of the hollow fibers and the size of the sphere imprints are coincident, because through these is that they retain a particular macromolecule whose size coincides with the pore size of the hollow fibers.

Note that the first (9) and second filter (11) has the same diameter and close the ends of the cylindrical container (10). Both the first filter (9) and second filter (11) have an input, through which the sample supplies uniformly to the filter and passes the cylindrical container (10).

The cylindrical container (10), has a plurality of hollow porous fibers (13) arranged in parallel and vertically to its axis, obtained as described above.

The resulting fibers in the above described process, are attachable together to form a modular device (14), all modules (13) optionally having of the same device the same characteristics or the pore sizes of the second filters gradually decrease as the number of modules increases that forms the device.

The method of operating the filtering modular device (14) comprises:
a) Inserting through an input of the Module of a modular device a liquid containing the particles to be separated to a flow of 1101 m−2*h−1, the inflow is dispersed uniformly in the filter,
b) Allowing to flow through the interior of the hollow porous fibers the liquid containing the suspended particles, such that when the fluid flowing, the particulates suspended that match the imprinted cavities in the spheres and the porosities of the fibers are retained,
c) Reclaiming the inflow and terminating the method of operation or to continue as in a) with subsequent modules. These modules are reconfigurable because its filtering mesh is removable. The passage of larger particles will have an appropriate filter with higher hole diameter, and so on. Leaving on the surface of the filtering mesh the particle concentrate that not allowed to pass (for its size) and only allowing the smaller ones size.

1. A process for obtaining porous hollow fibers with molecularly imprinted spheres embedded therein, characterized by comprising the steps of:
a) injecting (1) simultaneously and independently a doping solution and a pore-forming solution in an extrusion die;
b) extruding or spinning hollow porous fibers;
c) coagulating the hollow porous fibers in a aqueous solution coming from step b, with a aqueous solution that is at a temperature in the range of 15° C.-25° C.;
d) coiling the flexible and hollow porous fibers coagulated in step c), let them dry and store in vacuum conditions.

2. The process of claim 1, wherein in step a) the doping solution containing a plurality of molecularly imprinted microspheres dissolved in a solvent such as: n-methyl pyrrolidone, concentrated or dilute acetone between 1-2 M and possible combinations between them.

3. The process of claim 1, wherein in step a) the pore-forming solution comprising Polyethersulfone (PES) and polyether glycol (PEG) dissolved in a ratio ranging from 1:2 to 1:3.

4. The process of claim 1, wherein in step a) each of the solutions are separately stored and the inflow of the extrusion die is regulated by pumps so that it reaches the ideal operating conditions of temperature in the range of 40° C.-70° C. and 20-30 Pascal of pressure for the extrusion conditions in the die.

5. The process of claim 1, wherein in step b) the extrusion is performed with an outflow of hollow fibers equal to the injection flow of the doping solution and pore-forming solution.

6. A filtrating module comprising therein a plurality of hollow porous fibers obtained according to the process of claim 1, wherein the hollow porous fibers are in parallel and arranged longitudinally within a cylindrical container, the fibers are held at one end to a first filter that closes the upper end of the cylindrical container and closing the free end of the fibers is a second filter.

7. The module according to claim 6, wherein the pore size of the first filter is preferably higher than the pore size of the second filter.

8. The module according to claim 6, wherein the pore size of the first filter is equal to the pore size of the second filter.

9. The module according to claim 6, wherein the pore size of the second filter is less than the pore size of the hollow fibers and the imprints of the spheres embedded in the hollow fibers.

10. The module according to claim 6, wherein the pore size of the hollow fibers and the imprints of the spheres are equal.

11. A modular device formed by the filtrating module according to claim 6, wherein the filtering modules are attached together to form the modular device.

12. The modular device according to claim 11, wherein the filtrating modules have the same characteristics.

13. The modular device according to claim 11, wherein the filtrating modules share a filter, so that the second filter of a module corresponds to the first filtrating module that follows it.

14. The modular device according to claim 11, wherein the pore sizes of the second filters of each filtrating module preferably and gradually decrease as the number of modules increases that form the device.

15. A method of operating the a modular device comprising filtering modules attached together to form the modular device, wherein each filtering module comprises a filter having hollow porous fibers, the method comprising the steps of:
a) inserting through an input of a first of the filtering modules of the modular device an inflow of liquid containing particles to be separated, wherein the inflow is dispersed uniformly in the filter of the first of the filtering modules,
b) allowing to flow through the interior of the hollow porous fibers the liquid containing the suspended particles, such that when the fluid flowing, the particulates suspended that match the imprinted cavities in the spheres and the porosities of the fibers are retained,
c) reclaiming the inflow and terminating the method of operation or to continue as in a) with subsequent modules.
